(19) United States
(12) Patent Application Publication
(10) Pub. No.: US 2024/0010626 A1
(43) Pub. Date: Jan. 11, 2024

(54) METHOD FOR PRODUCING CARBONYLAMINOFURANS

(71) Applicant: Bayer Aktiengesellschaft, Leverkusen (DE)

(72) Inventor: Sergii PAZENOK, Leichlingen (DE)

(21) Appl. No.: 18/247,795

(22) PCT Filed: Oct. 4, 2021
(86) PCT No.: PCT/EP2021/077319

§ 371 Date: Apr. 4, 2023

(30) Foreign Application Priority Data
Oct. 6, 2020 (EP) 20200255.6

(51) Int. Cl. C07D 307/68

(52) U.S. Cl. CPC C07D 307/68

(57) ABSTRACT

The present invention relates to a novel method for preparing carbonylaminofurans of the general formula (I).

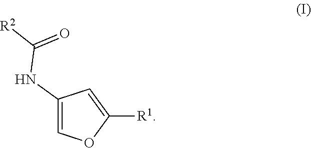

The present invention relates to a novel method for preparing carbonylaminofurans of the general formula (I).

4-Acylamino and alkoxycarbonylaminofurans of the general formula (I) (especially R¹=COOMethyl, R²=OtButyl) are important precursors of agrochemical active ingredients (cf. WO2018/228985) and pharmaceutical active ingredients (e.g., DNA binding agents: Woods, Craig R. et al. Bioorganic & Medicinal Chemistry Letters, 12(18), 2647-2650; 2002).

4-Acylaminofurans of the general formula (I) serve as starting material for the preparation of tetrahydro- and dihydrofurancarboxylic acids and esters. Hitherto, these compounds of the formula (I) have been prepared by a multi-stage synthesis including bromination, dehalogenation, and a coupling reaction (see F. Brucoli, et al. Bioorganic & Medicinal Chemistry, 20(6), 2019-2024; 2012).

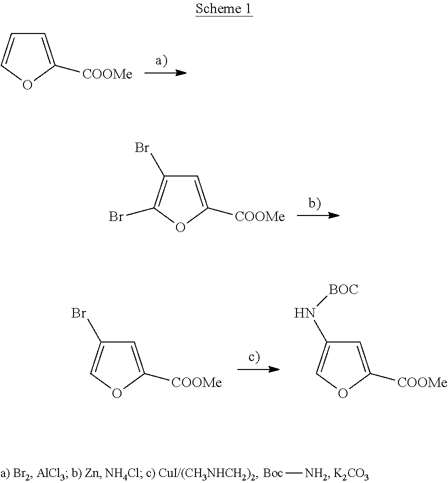

The synthesis mentioned above has a lot of disadvantages, such as low atom economy (bromination and debromination), use of heavy metals such as zinc and use of expensive reagents such as Boc-amine. The method described in Bioorganic & Medicinal Chemistry, 20(6), 2019-2024; 2012 furthermore requires the use of metal-containing (for example copper(I) iodide) catalysts.

These disadvantages render the method for preparing compounds of the general formula (I) uneconomic and therefore very expensive.

F. Wolter et al in (Organic Letters, 11(13), 2804-2807; 2009) describes another method for preparing aminofurans of the general formula (I), specifically via a Curtius rearrangement of 2-methyl furan-2,4-dicarboxylate using (PhO₃)₂P(O)N₃. This method is unsuitable for industrial applications due to the highly explosive properties of organic azides.

Several compounds of the general formula (I), for example where R¹=CF₃ and R²=NHAryl, have been described in EOC 2018, 3853-3861. However, this compound was detected in a mixture of several components.

In light of the prior art described above, the object of the present invention is to find a method for preparing the compounds of the general formula (I), which is cost-effective and which can be used on an industrial scale. It is also desirable to obtain these compounds with high yield and at high purity, such that they do not have to be subjected to any further complex purification.

The object described above of a simple, cost-effective and industrial preparation is achieved by a method for preparing compounds of the general formula (I)

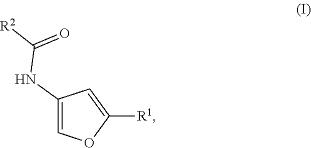

in which
R¹ is CF₃, CF₂H, C₂F₅, CF₂C₁, —COO—(C₁-C₆)-alkyl, COOH,
R² is H, (C₁-C₆)-alkyl, Cl, F, CF₃, CF₂C₁, CCl₃, —O—(C₁-C₆)-alkyl, —O—(C₁-C₆)-alkylaryl, —COO—(C₁-C₆)-alkyl,
characterized in that in a first step compounds of the general formula (II)

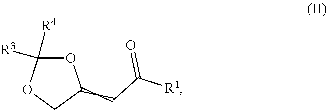

in which
R³ and R⁴ are each independently H and (C₁-C₆)-alkyl
and
R¹ has the definitions given above,
are converted with ammonia to compounds of the general formula (III)

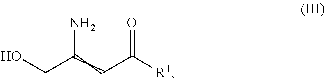

in which
R¹ has the definitions given above,
and in a second reaction step these are reacted in the presence of a dehydrating reagent to give compounds of the general formula (IV)

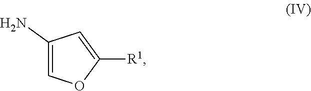

in which
R¹ has the definitions given above,
and in a third reaction step these are then reacted using an acylating reagent of the formula (V)

R²COX (V),
in which
R² is as defined above, and
X is F, Cl, Br, H₃CSO₂O, p-TolSO₂O, —OCOR²,
to give compounds of the general formula (I).

### Definitions

Alkyl means saturated straight-chain or branched hydrocarbyl radicals having the number of carbon atoms specified in each case, e.g. (C₁-C₆)-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Aryl means mono-, bi- or tricyclic aromatic or partially aromatic group having 6 to 14 carbon atoms, for example (but not restricted thereto) phenyl, naphthyl, tetrahydronaphthyl, indenyl and indanyl. The bonding to the superordinate general structure may be effected via any desired suitable ring member of the aryl radical. Aryl is preferably selected from phenyl, 1-naphthyl and 2-naphthyl. Particular preference is given to phenyl.

The compounds according to the invention are defined in general terms by the formula (I). Preferred substituents or ranges of the radicals given in the formulae mentioned above and below are illustrated hereinafter:

Preferred definitions of the radicals of the general formulae (I), (II), (III), (IV) and (V) are as follows:
R¹ is CF₃, CF₂H, CF₂C₁, C₂F₅, COOCH₃, COOC₂H₅,
R² is H, —(C₁-C₄)-alkyl, Cl, CF₃, CF₂C₁, CCl₃, —O—(C₁-C₄)-alkyl, —O—CH₂-phenyl, COOCH₃, COOC₂H₅,
R³ and R⁴ are each independently H or CH₃,
X is F, Cl, —OCOR², H₃CSO₂O, p-TolSO₂O.

Particularly preferred definitions of the radicals of the general formulae (I), (II), (III), (IV) and (V) are as follows:
R¹ is COOCH₃, COOC₂H₅,
R² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, CF₃, —O-methyl, —O-ethyl, —O-propyl, —O-1-methylethyl, —O-butyl, —O-1-methylpropyl, —O-2-methylpropyl, —O-1,1-dimethylethyl, —O-pentyl, —O-1-methylbutyl, —O-2-methylbutyl, —O-3-methylbutyl, —O-2,2-dimethylpropyl, —O-1-ethylpropyl, COOCH₃,
R³ and R⁴ are each independently H or CH₃,
X is Cl, —OCOR², H₃CSO₂O.

Especially preferred definitions of the radicals of the general formulae (I), (II), (III), (IV) and (V) are as follows:
R¹ is COOCH₃, COOC₂H₅,
R² is H, CH₃, CF₃, —OCH₃, —OC₂H₅, (CH₃)₃CO—, CCl₃, COOCH₃, —O—CH₂-phenyl,
R³ and R⁴ are CH₃,
X is Cl, —OCOR².

Most preferred definitions of the radicals of the general formulae (I), (II), (III), (IV) and (V) are as follows:
R¹ is COOCH₃, COOC₂H₅,
R² is CF₃, —OCH₃, —OC₂H₅, (CH₃)₃CO—, CCl₃, COOCH₃, —O—CH₂-phenyl
R³ and R⁴ are CH₃,
X is Cl.

The reaction sequence for preparing compounds of the formula (I) is shown in Scheme 2:

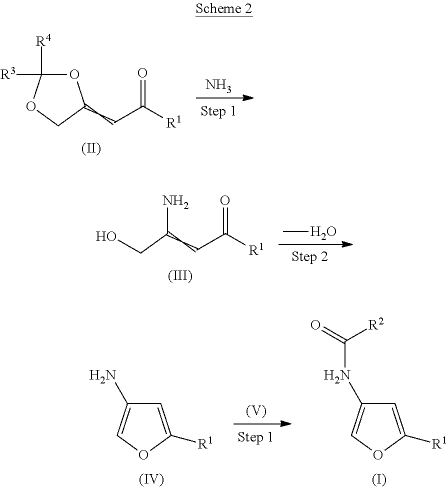

The compounds of the formula (II) react with ammonia to form the compounds of the general formula (III), which then in the second reaction step are converted to compounds of the general formula (IV) with elimination of water and are then reacted with an acylating reagent of the general formula (V) to give compounds of the general formula (I).

Step 1

The compounds of the formula (II) react with ammonia to form the compounds of the general formula (III).

The synthesis of the compounds of the general formula (II) and (III), in which R¹, R³ and R⁴ have the definitions specified above, is known. These compounds can be prepared by the method known from WO 2011/073100, WO 2011/073101 and European Journal of Organic Chemistry (2018), 2018(27-28), 3853-3861.

By way of example, the following compounds of the formula (II) may be mentioned:

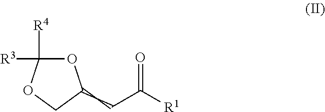

3-(2,2-dimethyl-1,3-dioxolan-4-ylidene)-1,1,1-trifluoropropan-2-one
3-(1,3-dioxolan-4-ylidene)-1,1,1-trifluoropropan-2-one
methyl 3-(2,2-dimethyl-1,3-dioxolan-4-ylidene)-2-oxopropanoate
ethyl 3-(2,2-dimethyl-1,3-dioxolan-4-ylidene)-2-oxopropanoate

By way of example, the following compounds of the formula (III) may be mentioned:

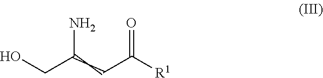

4-amino-1,1,1-trifluoro-5-hydroxypent-3-en-2-one
4-amino-1,1-difluoro-5-hydroxypent-3-en-2-one
4-amino-1,1,1-trichloro-5-hydroxypent-3-en-2-one
methyl 4-amino-5-hydroxy-2-oxopent-3-enoate
ethyl 4-amino-5-hydroxy-2-oxopent-3-enoate

Step 2

In the second reaction step, the compounds of the formula (III) are cyclized. The ring closure takes place in the presence of a dehydrating reagent such as SOCl₂, POCl₃, PCl₃, phosgene, diphosgene, triphosgene, ClCOCOCl, (CF₃CO)₂, P₄O₁₀, SO₂F₂, trimethyl orthoformate and triethyl orthoformate and HCl. Preferred reagents are SOCl₂, POCl₃, oxalyl chloride, phosgene and HCl.

The molar ratio of the compound of the formula (III) to the cyclization reagents is in the range of about 1:0.1 to 1:5, preferably from 1:0.5 to 1:2.

Reaction step 2 is usually carried out in a temperature range of 0° C. to 40° C. and optionally in the presence of a solvent or diluent. The reaction is preferably carried out in a solvent at approximately room temperature (RT).

Preferred solvents are methanol, ethanol, isopropanol, butanol, acetonitrile, N,N-dimethylacetamide, toluene, chlorobenzene, ethyl acetate, isopropyl acetate.

In the reaction with SOCl₂, POCl₃, PCl₃, phosgene, diphosgene, triphosgene, ClCOCOCl, the compounds of the general formula (IV) are obtained in the form of their HCl salts.

The salt-free form may be obtained by treating the salt with a base, for example triethylamine in ethyl acetate (see example 2).

By way of example, the following compounds of the formula (IV) may be mentioned:

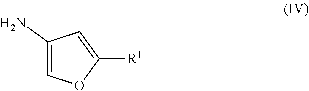

methyl 4-aminofuran-2-carboxylate hydrochloride/methyl 4-aminofuran-2-carboxylate
ethyl 4-aminofuran-2-carboxylate hydrochloride/ethyl 4-aminofuran-2-carboxylate
4-amino-2-trifluoromethylfuran hydrochloride

Step 3

In the third reaction step, the compounds of the formula (III) are acylated. The acylation is carried out with a reagent of the formula (V). The following compounds of the formula (V) may be mentioned by way of example: acetyl chloride, trichloroacetyl chloride, trifluoroacetyl chloride or anhydride, methyl oxalyl chloride, methyl chloroformate, tert-butyl chloroformate, benzyl chloroformate, Boc-anhydride.

The molar ratio of the compound of the general formula (IV) to the compound of the general formula (V) is in the range from about 1:0.9 to 1:2, preferably 1:1 to 1:1.5.

The acylation can be carried out with or without base. It may be considered surprising that it is also possible to use the salts (especially HCl salts) of the compounds of the general formula (IV) for the acylation step. If a base is used, the molar ratio of the compounds of the general formula (IV) to the base is from 1:0.5 to 1:3. Organic or inorganic compounds are suitable as bases.

Organic bases are: triethylamine, tributylamine, Hünig's base, pyridines, alkylpyridines, dimethylcyclohexylamine. Preferred bases are triethylamine, Hünig's base, 2-methyl-5-ethylpyridine, 3-picoline, dimethylcyclohexylamine.

Possible inorganic bases are: potash, Na₂CO₃, NaOAc.

Reaction step 3 is usually carried out in a temperature range of 10° C. to 40° C. and optionally in the presence of a solvent or diluent. The reaction is preferably carried out in a solvent at approximately room temperature (RT).

Preference is given to solvents such as toluene, chlorobenzene, acetonitrile, ether, dimethylacetamide, ethyl acetate, isopropyl acetate, dichloromethane. The compounds of the formula (I) are isolated by filtration of the product or extraction with an organic solvent (see examples).

Elucidation of the Methods and Intermediates

### EXAMPLES

The present invention is elucidated in more detail by the examples that follow, without restriction of the invention thereto.

Measurement Methods

The products were characterized by ¹H/¹³C-NMR spectroscopy and/or LC-MS (Liquid Chromatography Mass Spectrometry).

The NMR spectra were determined using a Bruker Avance 400 fitted with a flow probe head (volume 60 μl). In individual cases, the NMR spectra were measured with a Bruker Avance II 600.

### Example 1

Methyl 4-aminofuran-2-carboxylate hydrochloride (HCl salt of the compounds of the formula (IV)).

15.9 g (0.1 mol) of methyl 4-amino-5-hydroxy-2-oxopent-3-enoate were suspended in 50 ml of methanol and the mixture was cooled to 0° C. 17.7 g (0.15 mol) of SOCl₂ were added thereto at 0° C. over 2 hours. The mixture was stirred at 10° C. for a further 5 hours and the precipitate was filtered off, washed with 5 ml of methanol and dried. This gave 16.8 g, 95% of pale beige crystals.

¹H-NMR (400 MHz, CDCl₃): δ 10.07 (3H, s, br.); 8.10 (1H, d); 7.32 (1H, d); 3.83 (3H, s) ppm.

¹³C-NMR158.0 (s); 143.6 (s); 140.2 (d); 121.8 (s); 114.5 (d); 52.3 (q) ppm.

### Example 2

Conversion of methyl 4-aminofuran-2-carboxylate hydrochloride (salt of the formula (IV)) to methyl 4-aminofuran-2-carboxylate (salt-free product of the formula (IV)) 9.2 g of methyl 4-aminofuran-2-carboxylate hydrochloride were suspended in 50 ml of ethyl acetate and g of Et 3 N were added. The mixture was stirred at RT for 3 hours, the precipitate was filtered off and ethyl acetate fully concentrated under vacuum. This gave 6.96 g, 95% of beige crystals.

¹H-NMR (400 MHz, CDCl₃): δ: 7.24 (1H, d); 6.8 (1H, d); 4.3 (2H, s) 3.75 (3H, s) ppm.

### Example 3

Methyl 4-[(2,2,2-trifluoroacetyl)amino]furan-2-carboxylate 0.5 g of methyl 4-aminofuran-2-carboxylate hydrochloride were suspended in 15 ml of ethyl acetate and 1 g of (CF₃CO)₂O were added at 10° C. The mixture was stirred at RT for 5 hours and the precipitate was filtered off. This gave 0.55 mg of the product as a solid.

¹H-NMR (400 MHz, CDCl₃): δ 11.76 (1H, s, br.); 8.26 (1H, d); 7.24 (1H, d); 3.76 (3H, s) ppm.

¹³C-NMR158.2 (s); 154.1 (s, q); 142.5 (s); 137.4 (d); 124.7 (s); 115.8 (s); 112.1 (d); 52.3 (q) ppm.

### Example 4

Methyl 4-[(2-methoxy-2-oxoacetyl)amino]furan-2-carboxylate

0.5 g of methyl 4-aminofuran-2-carboxylate hydrochloride were suspended in 15 ml of ethyl acetate and 0.5 g of methyl oxalyl chloride were added at 10° C. The mixture was stirred at RT for 15 hours and the precipitate was filtered off. This gave 0.5 g (79%) of product.

Mass spectra m/z 227.

¹H-NMR (400 MHz, CDCl₃): δ 11.56 (1H, s, br.); 8.32 (1H, d); 7.36 (1H, d); 3.82 (3H, s), 3.32 (3H, s) ppm.

### Example 5

Methyl 4-(methoxycarbonylamino)furan-2-carboxylate

0.5 g of methyl 4-aminofuran-2-carboxylate hydrochloride were suspended in 15 ml of ethyl acetate and 0.5 g of methyl chloroformate were added at 10° C. The mixture was stirred at RT for 30 minutes and 0.5 g of NEt₃ were added in portions. The mixture was stirred at RT for 10 hours and diluted with 30 ml of ethyl acetate. The organic phase was washed with water and evaporated. This gave 0.54 g of product.

¹H-NMR (400 MHz, CDCl₃): δ 9.82 (1H, s, br.); 7.99 (1H, d); 7, 15 (1H, d); 3.86 (3H, s), 3.73 ppm

### Example 6

Methyl 4-(benzyloxycarbonylamino)furan-2-carboxylate

This was carried out as described in example 5 but taking 1.5 eq of benzyl chloroformate.

Yield 96%; m/z 275.

¹H-NMR (400 MHz, CDCl₃): δ 9.85 (1H, s, br.); 7.95 (1H, d); 7.4-7.15 (5H, m); 7.2 (1H, d), 5.2 (2H, s) 3.75 (3H, s) ppm.

### Example 7

Methyl 4[(2,2,2-trichloroacetyl)amino]furan-2-carboxylate

This was carried out as described in example 4 but taking 1.2 eq of CCl₃COCl.

Yield 88%; m/z 286.

¹H-NMR (400 MHz, CDCl₃): δ 11.2 (1H, s, br.); 8.45 (1H, d); 7, 45 (1H, d); 3.80 (3H, s), 3.73 ppm

1. A method for preparing compounds of general formula (I) 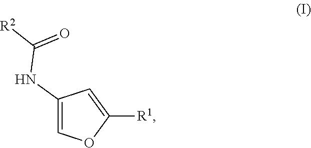
in which
R¹ is CF₃, CF₂H, C₂F₅, CF₂C₁, —COO—(C₁-C₆)-alkyl, COOH,
R² is H, (C₁-C₆)-alkyl, Cl, F, CF₃, CF₂C₁, CCl₃, —O—(C₁-C₆)-alkyl, —O—(C₁-C₆)-alkylaryl, —COO—(C₁-C₆)-alkyl,
characterized in that compounds of general formula (II) 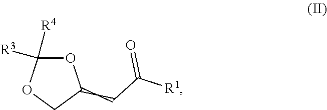
in which
R³ and R⁴ are each independently H and (C₁-C₆)-alkyl
and
R¹ has the definitions given above,
are converted with ammonia to compounds of general formula (III) 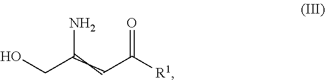
in which
R¹ has the definitions given above,
and these are reacted in the presence of a dehydrating reagent to give compounds of general formula (IV) 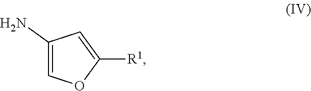
in which
R¹ has the definitions given above,
and these are then reacted using an acylating reagent of formula (V)
R²COX (V),
in which
R² is as defined above, and
X is F, Cl, Br, H₃CSO₂O, p-TolSO₂O, —OCOR²,
to give compounds of the general formula (I).

2. The method according to claim 1, characterized in that the definitions of the radicals of the compounds of the general formulae (I), (II), (III), (IV) and (V) are as follows:
R¹ is CF₃, CF₂H, CF₂C₁, C₂F₅, COOCH₃, COOC₂H₅,
R² is H, —(C₁-C₄)-alkyl, Cl, CF₃, CF₂C₁, CCl₃, —O—(C₁-C₄)-alkyl, —O—CH₂-phenyl, COOCH₃, COOC₂H₅,
R³ and R⁴ are each independently H or CH₃,
X is F, Cl, —OCOR², H₃CSO₂O, p-TolSO₂O.

3. The method according to claim 1, characterized in that the definitions of the radicals of the compounds of the general formulae (I), (II), (III), (IV) and (V) are as follows:
R¹ is COOCH₃, COOC₂H₅,
R² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, CF₃, —O-methyl, —O-ethyl, —O-propyl, —O-1-methylethyl, —O— butyl, —O-1-methylpropyl, —O-2-methylpropyl, —O-1,1-dimethylethyl, —O-pentyl, —O-1-methylbutyl, —O-2-methylbutyl, —O-3-methylbutyl, —O-2,2-dimethylpropyl, —O-1-ethylpropyl, COOCH₃,
R³ and R⁴ are each independently H or CH₃,
X is Cl, —OCOR², H₃CSO₂O.

4. The method according to claim 1, characterized in that the definitions of the radicals of the compounds of the general formulae (I), (II), (III), (IV) and (V) are as follows:
R¹ is COOCH₃, COOC₂H₅,
R² is H, CH₃, CF₃, —OCH₃, —OC₂H₅, (CH₃)₃CO—, CCl₃, COOCH₃, —O—CH₂-phenyl,
R³ and R⁴ are CH₃,
X is Cl, —OCOR².

5. The method according to claim 1, characterized in that the definitions of the radicals of the compounds of the general formulae (I), (II), (III), (IV) and (V) are as follows:
R¹ is COOCH₃, COOC₂H₅,
R² is CF₃, —OCH₃, —OC₂H₅, (CH₃)₃CO—, CCl₃, COOCH₃, —O—CH₂-phenyl
R³ and R⁴ are CH₃,
X is Cl.

6. The method according to claim 1, characterized in that from 1:0.1 to 1:5 equivalents of cyclization reagent are used, based on the compounds of the general formula (III).

7. The method according to claim 1, characterized in that the cyclization reagent is SOCl₂, POCl₃, oxalyl chloride, phosgene or HCl.

8. The method according to claim 1, characterized in that from 1:0.9 to 1:2 equivalents of the compound of the general formula (IV) are used, based on compounds of the general formula (V).

9. The method according to claim 1, characterized in that one of triethylamine, Hünig's base, 2-methyl-5-ethylpyridine, 3-picoline and dimethylcyclohexylamine is the acylating reagent.
